(19) United States
(12) Patent Application Publication
(10) Pub. No.: US 2026/0069228 A1
(43) Pub. Date: Mar. 12, 2026

(54) SYSTEMS AND METHODS FOR MEASURING A TRACT LENGTH DURING AN INTRAHEPATIC PORTOSYSTEMIC SHUNT PROCEDURE

(71) Applicant: Baylis Medical Technologies Inc., Mississauga (CA)

(72) Inventors: Amanda Centazzo-Colella, Bowmanville (CA); Amanda Hartley, Caledon (CA); Patrick Ryan, Toronto (CA)

(21) Appl. No.: 19/388,031

(22) Filed: Nov. 13, 2025

Related U.S. Application Data
(63) Continuation of application No. PCT/IB2024/054276, filed on May 2, 2024.
(60) Provisional application No. 63/500,006, filed on May 4, 2023.

(51) Int. Cl. A61B 6/50; A61B 6/00; A61B 6/12; A61M 25/01

(52) U.S. Cl. CPC A61B 6/504; A61B 6/12; A61B 6/481; A61M 25/0108

(57) ABSTRACT

A medical device assembly and method of using the assembly are disclosed for measuring a tract length during a transvascular procedure such as a Transjugular Intrahepatic Portosystemic Shunt (TIPS) procedure, or a Direct Portosystemic Intrahepatic Shunt (DIPS) procedure. The present invention provides a telescoped assembly for injecting contrast at a first location and a second location, the first and second locations on opposing sides of the tract, while substantially maintaining the position of the assembly, and without removing one or more inner devices from the assembly, thereby reducing procedure time.

### TECHNICAL FIELD

The disclosure relates to medical devices, and more particularly to devices and methods for measuring a distance within a human body during a medical procedure.

### BACKGROUND

For patients suffering from portal hypertension, a shunt may be placed from the hepatic vein to the portal vein, which allows blood flow to bypass the liver and alleviates the portal pressure. Creation of the shunt is done percutaneously and uses specialized catheters and wires.

During a Transjugular Intrahepatic Portosystemic Shunt (TIPS) procedure, once a tract has been created via a puncture into the portal vein, a stent is deployed to dilate the tract and maintain patency of the shunt. The stent generally spans the distance from the portal vein, through the tract, and terminates in the inferior vena cava (IVC).

Typically, a specialized sizing catheter with radiopaque marker bands is inserted across the tract and fluoroscopy is used to measure the tract length. Once this sizing catheter is placed across the tract, contrast is injected into the hepatic vein to visualize the tract and the hepatic vein/IVC confluence. This is generally referred to as a venogram, cavogram or tractogram. The radiopaque marker bands are counted in order to select an appropriate stent size.

### BRIEF DESCRIPTION OF THE DRAWINGS

In order that the invention may be readily understood, embodiments of the invention are illustrated by way of examples in the accompanying drawings, in which:

FIG. 1A is an illustration of the anatomy of a liver and surrounding blood vessels showing an elongate medical device across a tract from the hepatic vein, through the liver, to the portal vein;

FIG. 1B is an illustration of the anatomy of a liver and surrounding blood vessels showing a stent extending from the portal vein to the inferior vena cava;

FIGS. 2A and 2B are illustrations of a telescoping assembly used to perform a TIPS procedure in accordance with an embodiment of the present invention;

FIG. 3 is an illustration of a steerable sheath in accordance with an embodiment of the present invention;

FIG. 4 is an illustration of a dilator in accordance with an embodiment of the present invention;

FIG. 5 is an illustration of a catheter in accordance with an embodiment of the present invention;

FIG. 6A is illustration of the distal end of the telescoping assembly in accordance with an embodiment of the present invention;

FIG. 6B is illustration of the distal end of the telescoping assembly in accordance with an alternative embodiment of the present invention;

FIG. 7 is an illustration of a telescoping assembly injecting contrast fluid in accordance with an embodiment of the present invention;

FIGS. 8A and 8B are illustrations of the proximal ends of devices of the telescoping assembly in accordance with an embodiment of the present invention;

FIGS. 9A-9C are illustrations of a device of the telescoping assembly in accordance with an alternative embodiment of the present invention;

FIG. 10 is an illustration of a telescoping assembly injecting contrast fluid in accordance with an alternative embodiment of the present invention;

FIGS. 11A-11D are illustrations of a device comprising measurement indicators in accordance with an embodiment of the present invention;

FIG. 12 discloses a method of facilitating a TIPS procedure in accordance with an embodiment of the present invention;

FIG. 13 discloses a method of facilitating a TIPS procedure in accordance with an alternative embodiment of the present invention;

FIG. 14 discloses a method of facilitating a TIPS procedure in accordance with a further alternative embodiment of the present invention.

In the drawings, like reference characters generally refer to the same parts throughout the different views. Also, the drawings are not necessarily to scale, emphasis instead being placed upon generally illustrating the various concepts discussed herein.

### DETAILED DESCRIPTION

In one broad aspect, embodiments of the present invention comprise a method of measuring a tract length during an intrahepatic portosystemic shunt procedure, the method comprising the steps of: visualizing a tract between a first blood vessel and a second blood vessel by injecting contrast fluid into at least one of the first blood vessel and the second blood vessel using an elongate device, contrast fluid injected into the first blood vessel is injected through a first opening of the elongate device and contrast fluid injected into the second blood vessel is injected through a second opening of the elongate device; and determining a tract length using a visual indicator.

As a feature of this broad aspect, in some embodiments, the step of visualizing the tract comprises injecting contrast fluid into the first blood vessel and the second blood vessel substantially concurrently.

As another feature of this broad aspect, in some embodiments, the visual indicator is associated with the elongate device.

As another feature of this broad aspect, in some embodiments, the visual indicator is associated with a second elongate device, the second elongate device being received within and moveable relative to the elongate device.

As another feature of this broad aspect, in some embodiments, the visual indicator comprises a plurality of radiopaque marker bands.

As another feature of this broad aspect, in some embodiments, the method, further comprises a step of selecting a stent suitable to be inserted between the first blood vessel and the second blood vessel based on the tract length.

As another feature of this broad aspect, in some embodiments, the elongate device comprises an elongate member defining a primary lumen and a secondary lumen, the primary lumen terminates at the first opening which is defined by an inner wall and located at a distal end of the elongate member, and the secondary lumen terminates at the second opening which is defined by an outer wall and located a linear distance proximal to the first opening.

As another feature of this broad aspect, in some embodiments, the visual indicator is associated with a proximal end of the second elongate device, and the step of determining the tract length comprises moving the elongate device, while substantially maintaining a position of the second elongate device, and measuring a distance a proximal end of the elongate device has moved relative to the visual indicator.

As another feature of this broad aspect, in some embodiments, the first blood vessel is a portal vein and the second blood vessel is a hepatic vein.

In a further broad aspect, embodiments of the present invention comprise a method of measuring a tract length during an intrahepatic portosystemic shunt procedure using a first elongate device and a second elongate device, the second elongate device being received within and moveable relative to the first elongate device, the method comprising the steps of: positioning a distal end of the first elongate device in a first location within a patient's body; determining a starting position of a proximal end of the first elongate device relative to a proximal region of the second elongate device; while substantially maintaining a position of the second elongate device, retracting the distal end of the first elongate device to a second location such that the proximal end moves to a retracted position; and determining the tract length based on a distance between the starting position and the retracted position of the proximal end of the first elongate device.

As a feature of this broad aspect, in some embodiments, the proximal region of the second elongate device comprises graduated markings.

As another feature of this broad aspect, in some embodiments, the method further comprises the step of injecting contrast fluid at least at one of the first location and a second location.

As another feature of this broad aspect, in some embodiments, the first location is a first blood vessel, and the second location is a second blood vessel.

As another feature of this broad aspect, in some embodiments, the first blood vessel is a portal vein, and the second blood vessel is a hepatic vein.

In a further broad aspect, embodiments of the present invention comprise a medical device operable to inject fluid, the medical device comprising an elongate member, the elongate member comprising: an inner wall and an outer wall; a first opening defined by a distal end of the elongate member; a primary lumen defined by the inner wall and terminating at the first opening; a second opening defined by the outer wall and located proximally of the first opening; a secondary lumen terminating at the second opening; and a visual indicator located at least between the first opening and the second opening; in use, fluid injected through the primary lumen exits from the first opening and fluid injected through the secondary lumen exits from the second opening.

As a feature of this broad aspect, in some embodiments, the secondary lumen is defined between the inner wall and the outer wall.

As a feature of this broad aspect, in some embodiments, the secondary lumen is located substantially within the primary lumen.

As another feature of this broad aspect, in some embodiments, the visual indicator comprises a plurality of radiopaque marker bands.

As another feature of this broad aspect, in some embodiments, the visual indicator comprises radiopaque ink.

As another feature of this broad aspect, in some embodiments, the second opening is located between about 3 centimetres and about 7 centimetres from the proximal opening.

For explanatory purposes, the systems and methods disclosed herein are generally described with reference to a TIPS procedure, where a tract is created from the hepatic vein to the portal vein. As would be apparent to one skilled in the art, the systems and methods described herein are also applicable to a direct intrahepatic portosystemic shunt (DIPS) procedure. Certain aspects of this disclosure are also applicable to other medical procedures as well beyond TIPS and DIPS.

At least part of a TIPS procedure can be performed using a telescoping assembly comprising a flexible radiofrequency (RF) device, a catheter, a dilator, and a steerable sheath. An example of such an assembly is disclosed in U.S. Pat. No. 11,324,548 B2, granted on May 10, 2022, to Baylis Medical. Other systems or assemblies may be used as well, and the invention is not limited in this regard. U.S. Pat. No. 11,324,548 discloses creating a track using an RF device but does not include steps of measuring the tract length.

One method of determining the tract length during a TIPS procedure involves inserting a dedicated sizing catheter comprising radiopaque marker bands. Once the sizing catheter is in place, a physician uses a second catheter to inject contrast fluid to visualize the tract and the marker bands using fluoroscopy.

Contrast fluid may be injected into the hepatic vein or somewhere else in the tract, such that it fills both the tract and the IVC. Ideally, the portal vein, the tract, and the hepatic vein could be visualized. If a physician wants to inject contrast fluid into the portal vein, the distal end of the catheter would have to be advanced into the portal vein. The physician would then retract upwards to the tract or the hepatic vein and inject again. Any additional or unnecessary movements take extra time and may pose additional risk. The ability to inject contrast at two locations substantially without repositioning the catheter may be advantageous, as it would provide a better image to visualize the portal vein, the tract, and the hepatic vein.

With specific reference now to the drawings in detail, it is stressed that the particulars shown are by way of example and for purposes of illustrative discussion of certain embodiments of the present invention only. Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

A system or apparatus, as well as a method of use thereof, is described herein that is operable to measure a tract length during a TIPS procedure while mitigating one or more of the risks noted herein.

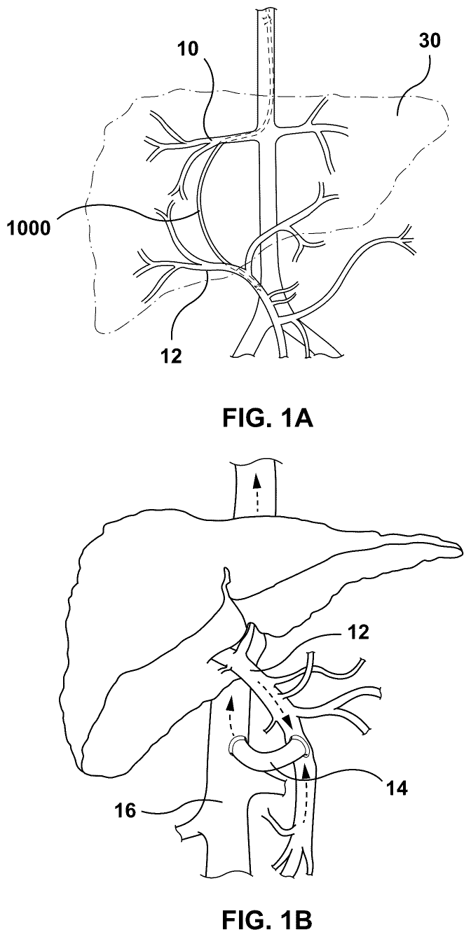

Referring to FIGS. 1A and 1B, a liver 30 and adjoining structures are shown. FIG. 1A shows a flexible, elongate medical device 1000 crossing a tract through liver tissue. The tract extends between a first blood vessel and a second blood vessel, where the first blood vessel is the hepatic vein 10 and the second blood vessel is the portal vein 12, for example during a TIPS procedure. FIG. 1B shows a shunt 14 extending from a third blood vessel to the second blood vessel, where the third blood vessel is the inferior vena cava 16 and the second blood vessel is the portal vein 12, for example during a DIPS procedure.

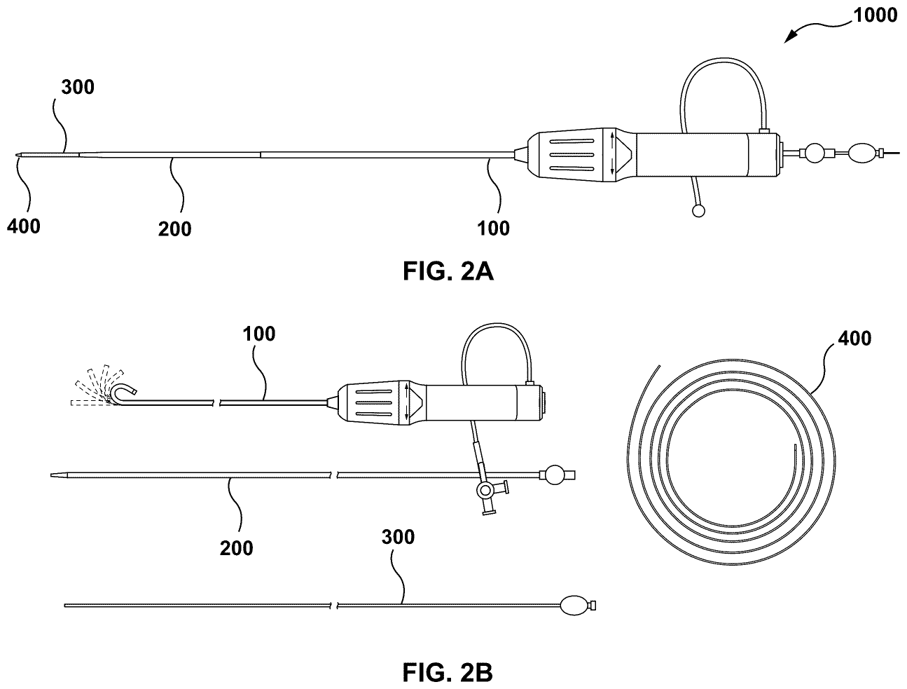

Referring to FIGS. 2A and 2B, in one embodiment of the present invention, the elongate medical device 1000 comprises a telescoping assembly 1000 for performing at least part of a TIPS procedure. In one example, the telescoping assembly 1000 comprises sheath 100 (which may also be referred to as a “steerable sheath”), dilator 200, catheter 300, and puncture device 400. In one embodiment, puncture device 400 is an RF guidewire, capable of delivering RF energy to cut through tissue. In other embodiments, puncture device 400 is a needle, stylet, or other guidewire that uses mechanical energy to puncture.

In one specific example, the telescoping assembly 1000 of the present invention comprises a 10 French (Fr) steerable sheath, a 10 Fr flexible dilator, a 4 Fr crossing catheter, and a 0.035″ RF Guidewire. In one such example, the telescoping assembly 1000 dilator 200 is received within the sheath 100, catheter 300 is received within the dilator 200, and puncture device 400 is received within the catheter 300, each component being in a telescoping arrangement and longitudinally moveable relative to the other devices. FIG. 2A shows the devices received within each other, and FIG. 2B shows the devices separate.

For descriptive purposes, any of the sheath, dilator, catheter, or RF wire may be referred to as a “device”, such as when describing modifications to or relationships between the components. For example, a device comprising marker bands means one of a sheath, catheter, dilator, or wire comprising marker bands. Devices may also be referred to as either an “inner device” or an “outer device” depending on the context. For example, when describing the sheath and catheter, the catheter may be positioned within the sheath, such that the catheter is referred to as the inner device, and the sheath is referred to as the outer device.

In some embodiments, each device of the telescoping assembly 1000 is separate and fully removable from all other devices. In other words, the devices may be assembled in any suitable combination depending on the application. For example, if a procedure requires more space within the sheath, then one or more of the other devices within the telescoping assembly, for example the dilator, may be removed. In addition, certain devices may be “backloadable” over other devices of the assembly. For example, a dilator may be advanced proximally or removed distally over top of a catheter, or the dilator may be removed from the assembly without first having to remove the catheter.

Such embodiments that allow for removal of one or more components of the assembly during a TIPS procedure may provide particular advantages. For example, if, during a procedure, one of the devices becomes problematic, the problematic device may be swapped or exchanged for a new device.

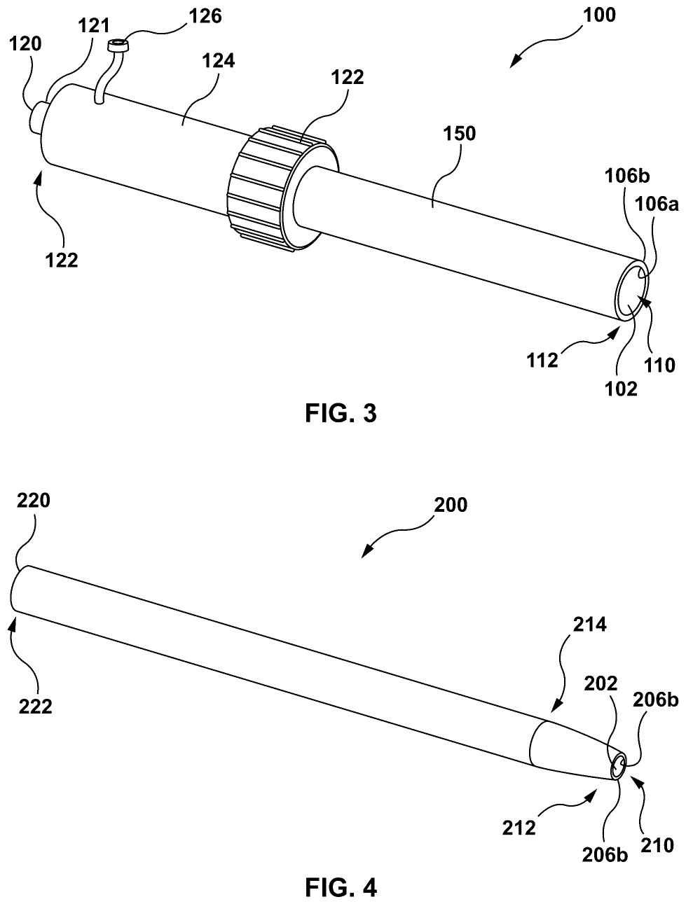

With reference to FIG. 3, according to one embodiment of the present invention, sheath 100 comprises a handle 124 and a shaft 150. Shaft 150 comprises a flexible elongate member having an inner wall 106a and outer wall 106b, together defining a primary lumen 102. Primary lumen 102 extends between a distal opening 110 at a distal end 112 and a proximal opening 120 at a proximal end 122. In one embodiment, handle 124 comprises a flush port 126, described herein, for injecting fluid through sheath primary lumen 102. Flush port 126 is in fluid communication with sheath primary lumen 102. Handle 124 further comprises an actuator 132 for controlling a deflection of the shaft near the distal end 112.

Primary lumen 102 is sized to receive one or more inner devices, for example, dilator 200, extending through distal opening 110 and proximal opening 120. Dilator 200 is configured to be insertable into primary lumen 102. Proximal opening 120 may also include a hemostatic valve 121 for creating a seal to prevent fluids from escaping.

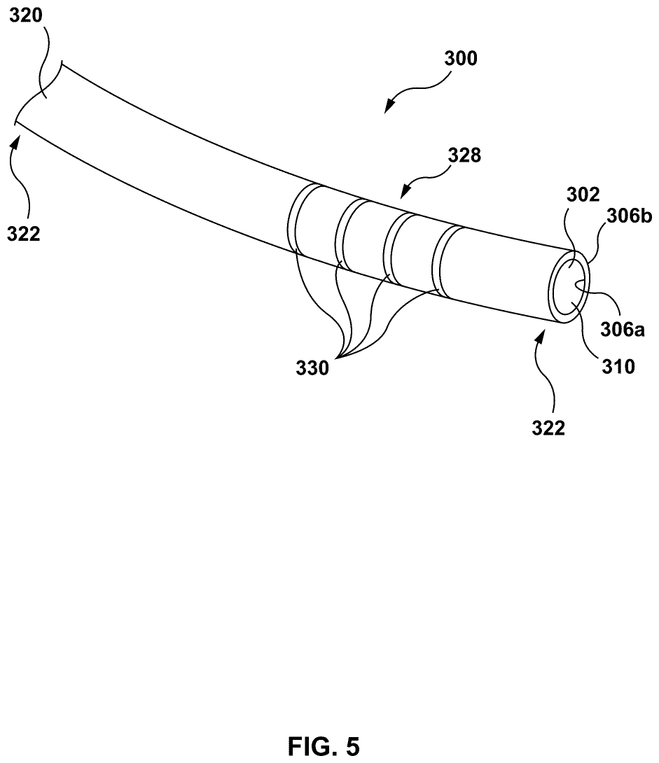

With reference to FIG. 4, according to one embodiment of the present invention, dilator 200 comprises an inner wall 206a and outer wall 206b, together defining a primary lumen 202. Primary lumen 102 extends between a distal opening 210 at a distal end 212 and a proximal opening 220 at a proximal end 222. Primary lumen 202 is sized to receive one or more inner devices, for example, catheter 300, extending through distal opening 210 and proximal opening 220. In some embodiments, distal end 212 includes a tapered portion 214.

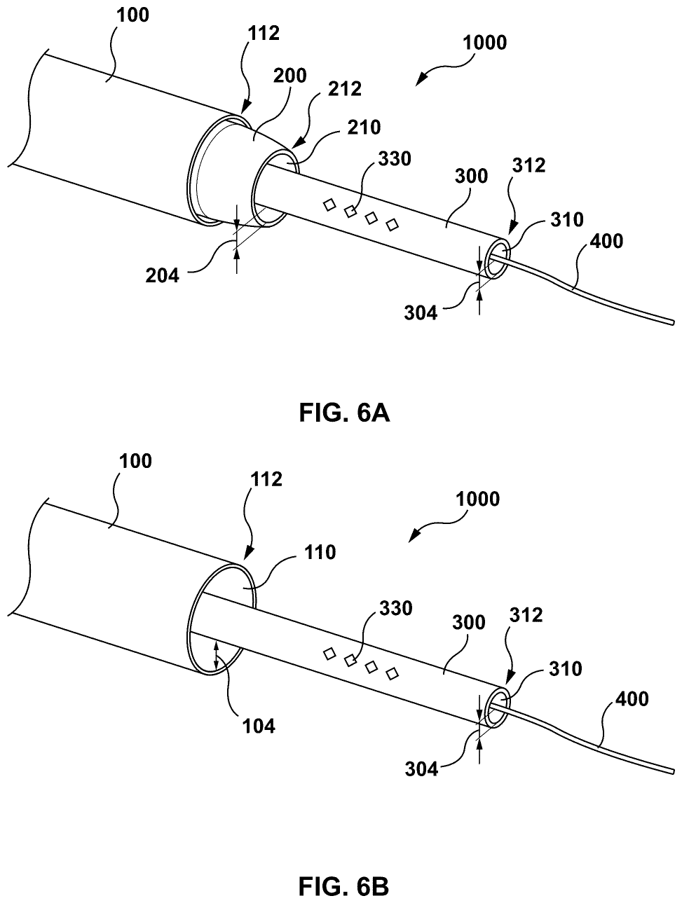

With reference to FIG. 5, according to one embodiment of the present invention, catheter 300 comprises an inner wall 306a and outer wall 306b, together defining a primary lumen 302. Primary lumen 302 extends between a distal opening 310 at a distal end 312 and a proximal opening 320 at a proximal end 322. Catheter primary lumen 302 is sized to receive one or more inner devices, for example, puncture device 400, extending through distal opening 310 and proximal opening 320. Catheter 300 is configured to be insertable into dilator 200, and puncture device 400 is configured to be insertable into catheter 300.

In one embodiment, catheter 300 comprises a visual indicator 328. In one example, visual indicator 328 comprises one or more marker bands 330, which may be any suitable means that are visible on fluoroscopy or other visualization method. In one embodiment, marker bands 330 comprise radiopaque marker bands affixed to the outer wall 306b. In another embodiment, visual indicator 328 comprise radiopaque ink.

In other embodiments, one or more marker bands 330 may be located on any of the devices capable of supporting them, for example the sheath 100, dilator 200, or puncture device 400.

In some embodiments, marker bands 330 are spaced at predetermined intervals, for example, each marker band 330 is spaced 2 cm apart. In another embodiment, marker bands 330 are arranged with a “datum” band that is spaced between a first marker band 330 and second marker band 330 and subsequent bands are spaced at regular intervals from the datum. Such an embodiment may require fewer marker bands. In use, a physician may estimate the required stent length if the tract length falls between two of the marker bands 330.

In one embodiment, puncture device 400 is and RF guidewire of the type disclosed in U.S. Pat. No. 9,510,900 B2, granted Dec. 6, 2016, to Baylis Medical, which is hereby incorporated by reference. In other embodiments, puncture device may be a stylet or a mechanical needle similar to known devices, such as a Colapinto needle.

### Injecting Contrast at Two Locations Using Two Devices

Current TIPS procedures typically involve using a single device, for example a catheter, to inject contrast at one or more desired vascular locations. Using a single device requires moving the device between various locations, for example injecting contrast in the hepatic vein, then moving the device through the tract, into the portal vein, and injecting contrast there. Some sizing catheters are not configured to inject contrast while an inner guidewire is located within its primary lumen. In other words, the guidewire needs to be removed from the sizing catheter so that contrast can be injected through it. The ability to inject contrast without having to remove the guidewire could be advantageous. Additionally, contrast could be injected through the sizing catheter at one location, and through the sheath at a second location, which could improve the imaging used to select a stent size.

There are several locations where contrast fluid may be injected during a TIPS procedure, such as the hepatic vein, the portal vein, the tract between the hepatic and portal veins, and near the HV/IVC confluence. In order to visualize the blood vessels and the tract, contrast fluid may need to be injected at multiple locations, which may necessitate moving a single device through which the contrast is injected, which is disadvantageous for several reasons. For example, additional device movements may prolong the procedure and have the potential to damage tissue. Additionally, the insertion and removal of a dedicated sizing catheter may prolong the procedure.

The present inventors have conceived of devices, assemblies and methods that allow for injecting contrast at two locations and for measuring the length of the tract between the hepatic and portal veins with fewer device movements and/or exchanges than are typically employed. The present invention is particularly advantageous as it avoids unnecessary movement or exchange of devices which prolong the procedure or could potentially damage the patient. Further, moving devices through the intrahepatic tract may be challenging due to the stiffness of liver tissue. Risks of tissue damage and prolonged procedures are mitigated by the present invention. Additionally, incorporating sizing features, for example radiopaque marker bands, on one or more of the devices may further obviate the need for an additional exchange of devices which may therefore reduce procedural time. In other words, using embodiments of the present invention, a single device may perform a task typically performed by two or more devices.

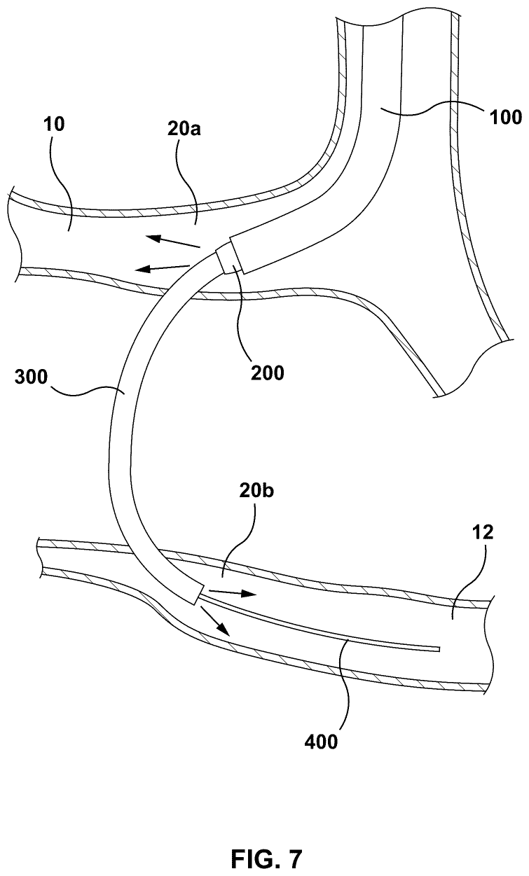

Referring to FIG. 6A, one embodiment of the telescoping assembly 1000 is shown in a telescoped arrangement. Catheter 300 and dilator 200 are configured such that catheter 300 has an outer diameter that is less than the inner diameter of dilator 200 and such that there is a gap 204 at the distal end 212 of the dilator 200. Similarly, the dimensions of the catheter 300 and puncture device 400 are configured such that puncture device 400 has an outer diameter that is less than the inner diameter of catheter 300, such that there is a gap 304 at the distal end 312 of the catheter 300.

Contrast fluid may be injected through dilator primary lumen 202 and catheter primary lumen 302 and exit at gap 204 and gap 304 respectively, e.g., out of distal openings 210 and 310. In such an embodiment, puncture device 400 does not need to be removed from catheter 300 in order for contrast to be injected through catheter primary lumen 302 and exit at distal opening 310. Two fluid injections at two separate locations can be performed while all four devices are in an assembled/telescoped configuration, i.e., puncture device 400 is within catheter 300, catheter 300 is within dilator 200, and dilator 200 is within sheath 100.

In some embodiments, puncture device 400 may be removed from telescoping assembly 1000, and gap 304 is equal to the inner diameter of catheter distal opening 310.

FIG. 6B shows the telescoping assembly 1000 with dilator 200 removed. Similar to the embodiment shown in FIG. 6A, there is a gap 104 created due to the dimensions of sheath 100 inner diameter and catheter 300 outer diameter, and fluid may be injected through gap 104 at sheath distal end 112. In one embodiment, sheath 100 and dilator 200 have similarly sized diameters. (Note, marker bands 330 are not shown to scale, or necessarily in the proper location along the shaft in order to measure a tract.)

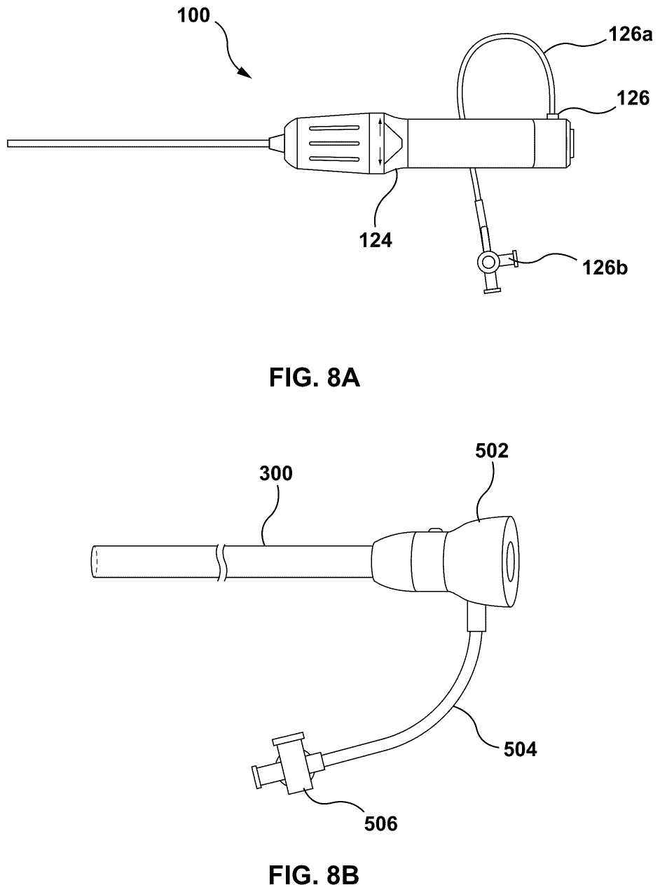

With reference to FIG. 7, telescoping assembly 1000 is shown in use during a TIPS procedure. Telescoping assembly 1000 extends across a tract created through a portion of the liver (not shown), from the hepatic vein 10 to the portal vein 12. In one example, sheath distal end 112 and dilator distal end 212 are located in the hepatic vein 10 and catheter distal end 312 is located in the portal vein 12. The arrows 20a and 20b in FIG. 7 represent the flow of contrast fluid through gaps 204 and 304 described above. Puncture device 400, once sufficiently tracked into the portal vein 12, may be used as a guidewire for at least part of the TIPS procedure. In other embodiments, puncture device 400 may be removed from the assembly (after the puncture through the liver) and any other suitable guidewire may be used, such there is a gap 304 between the catheter inner wall 302a and the guidewire.

As used herein, injecting contrast fluid into a first location means injecting fluid into a patient's body where the distal end (or other opening or aperture) of a device is located. Contrast fluid injected at one location may flow into one or more blood vessels. For example, contrast fluid injected into the hepatic vein may flow into one or more of the tract and the IVC. In some instances, injecting contrast fluid into the hepatic vein means injecting contrast fluid into the hepatic vein such that it flows into the IVC, and/or the HV/IVC confluence.

Similarly, references to injecting contrast fluid at a second location means that the distal end (or other opening or aperture) of the same device is in a different location than the first location, or it may refer to injection of fluid through an opening in a second device positioned at a different location. In either case, injecting into a second location is understood to refer to the location at which the fluid exits the device into the body, even though the fluid may thereafter flow into one or more of the same blood vessels as it flowed into after being injected at the first location.

To inject contrast fluid, additional components are connected to the proximal ends of the devices. In one embodiment, an injection device, for example a syringe, is operable to be connected to the proximal end of one or more devices of telescoping assembly 1000.

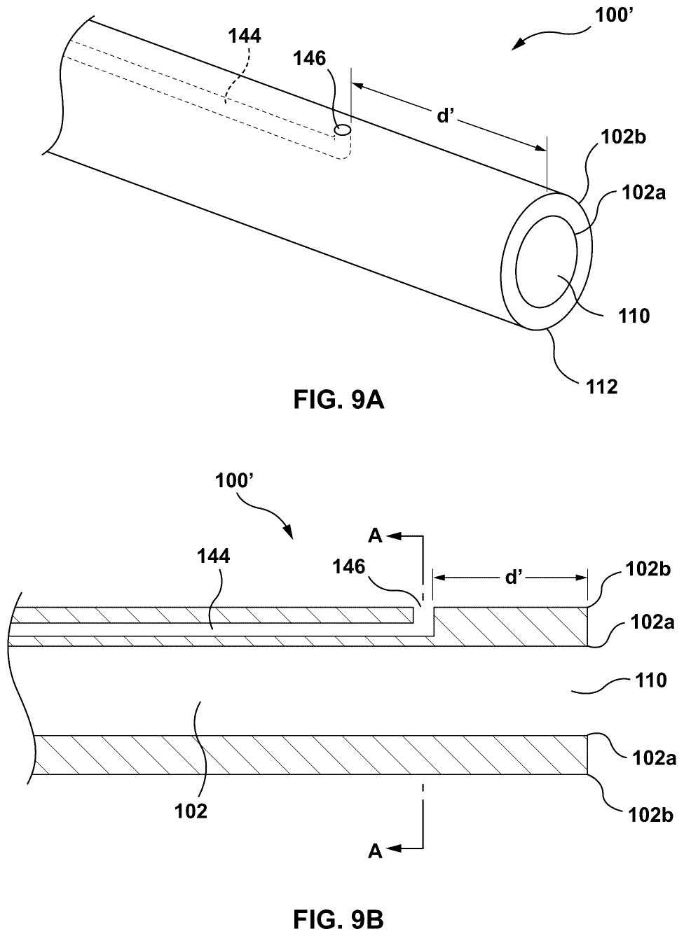

Referring to FIG. 8A, in one embodiment, sheath 100 comprises a handle 124 at the proximal end 122, handle 124 includes a flush port 126 for injecting fluids through sheath primary lumen 102. Flush port 126 is in fluid communication with sheath primary lumen 102. Flush port 126 comprises a flexible tube 126a and a valve 126b for connecting to an external device, for example a syringe, via luer fittings.

Referring to FIG. 8B, in one embodiment, hub 502 is coupled to the catheter proximal end 322. Hub 502 is coupled to flexible hose 504, which couples to valve 506, for example a stopcock. Valve 506 can be coupled to an injection device.

In other embodiments, (not shown), dilator 200 is coupled to hub 502 such that flid may be injected through dilator 200.

Injection device may be any device capable of injecting contrast fluid into the lumen of any one of the sheath, dilator, or catheter. Contrast fluid may be any injectable medium that is visible under an imaging modality. For example, contrast fluid may be a dye or fluid capable or making one or more blood vessels or tracts visible under known imaging techniques, for example iohexol. In other words, once contrast fluid is injected into a blood vessel, the blood vessels may be visualized under an imaging modality. Several images, or tractograms, may be taken using known means to visualize the tract. One skilled in the art will understand that fluids other than contrast fluid may also be used with one or more embodiments of the present invention.

In one embodiment, two injection devices are connected to the proximal ends of two devices, and contrast fluid may be injected through the two devices, thereby delivering contrast fluid to two locations while maintaining the position of the telescoping assembly 1000. In other words, contrast fluid may be injected at two locations without moving any of the devices.

In one specific example, a first injection device is operably coupled to the sheath 100 and a second injection device is operably coupled to the catheter 300. Contrast fluid can then be injected through sheath 100 and catheter 300 into hepatic vein 10 and portal vein 12 respectively. In other words, contrast fluid can be injected at two locations while maintaining the respective positions of each of the two devices. Further, in such an embodiment, it is possible to inject contrast fluid at two locations concurrently. After contrast fluid is injected, a tract measurement can be made by viewing a visual indicator associated with one of the devices.

### Injecting Fluid at Two Locations Using One Device

In another embodiment of the present invention, devices and methods are disclosed for injecting contrast fluid at two locations using a single device during a procedure such as TIPS.

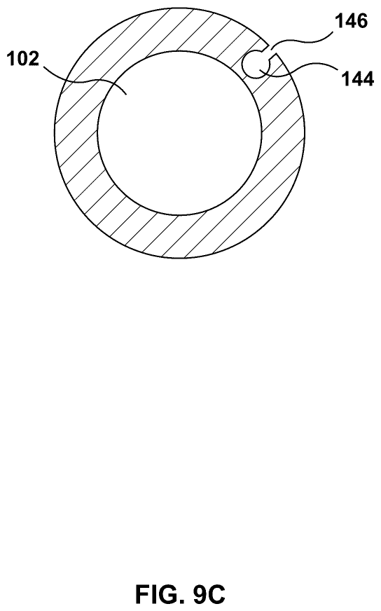

Referring to FIGS. 9A to 9C, in one embodiment, sheath 100′ comprises an inner wall 106a and an outer wall 106b defining primary lumen 102 that terminates at distal opening 110. Sheath 100′ also defines a secondary lumen 144, that extends from the proximal end 122 of sheath 100′, to an aperture 146, located a linear distance d′ from the distal end 112. Aperture 146 is defined by the outer wall 106b. In one embodiment, secondary lumen 144 may be located substantially between inner wall 106a and outer wall 106b, as shown in FIGS. 9B and 9C. In one embodiment, d′ is between about 3 cm and about 7 cm.

In another embodiment, a portion of secondary lumen 144 may be located substantially within the primary lumen 102 and may pass through inner wall 106a and outer wall 106b at the aperture 146 (not shown).

An injection device, described herein, is operably coupled to the proximal end of sheath 100′, such that the injection device is in fluid communication with secondary lumen 144, for example using a hub. Contrast fluid may then be injected through secondary lumen 144 exiting at aperture 146 to a location proximal of the distal end 112. d′ may be a distance such that that aperture 146 is located in a first location/first blood vessel and the distal opening 110 is located in a second location/second blood vessel. A second injection device is operably coupled to sheath 100′ at flush port 126, such that contrast fluid can be injected through the primary lumen 102 and exits at the distal opening 110.

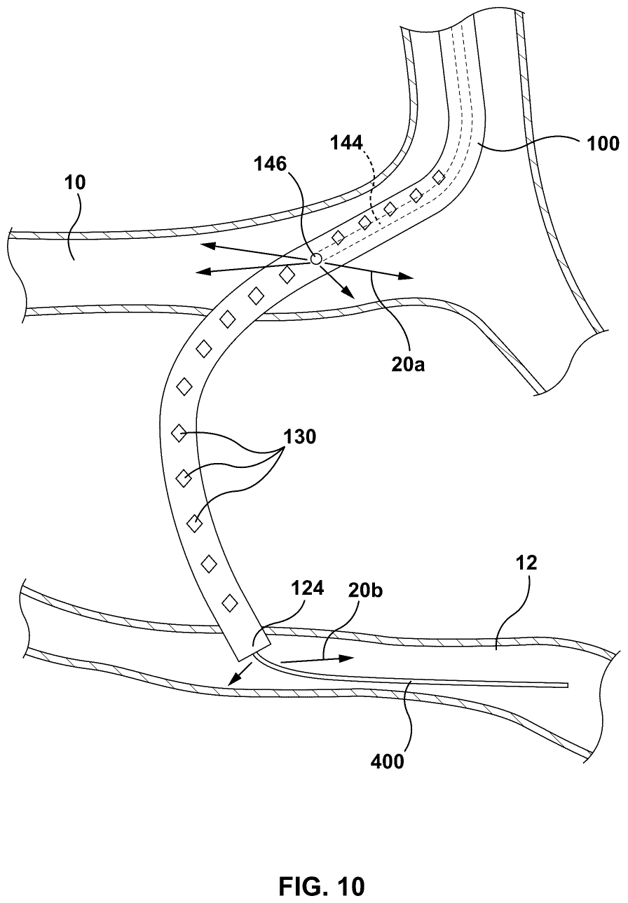

With specific reference to a TIPS procedure, as shown in FIG. 10, d′ may be of a length such that contrast fluid is injected in both the hepatic vein 10 and portal vein 12 without repositing sheath 100′. In other words, aperture 146 is located in the hepatic vein 10 and distal opening 110 is located in the portal vein 12. Further, contrast fluid may be injected through aperture 146 and distal opening 110 concurrently. The flow of contrast fluid to two locations using a single device is represented by the arrows 20a and 20b.

In one embodiment, sheath 100′ comprises a visual indicator 128, for example, marker bands 130, as described herein. Marker bands 130 may be located at least between distal opening 110 and aperture 146. In another embodiment marker bands 130 are located between the distal end 112 and continue proximate of aperture 146.

In another embodiment, a single injection device is operably coupled to the proximal end 122 of sheath 100 such that the injection device is capable of injecting fluid through both the primary lumen 102 and secondary lumen 144. In some embodiments, contrast fluid may be injected through primary lumen 102 and secondary lumen 144 concurrently.

In other embodiments, one of the catheter 300 or dilator 200 may define a secondary lumen and aperture described above. In still further embodiments, any device may comprise a visual indicator described herein. The visual indicator may extend from the distal end to a region proximal of aperture 146, such that in use during a TIPS procedure, visual indicator spans the distance from the portal vein 12 to the IVC 16. In other words, visual indicator may extend from a device distal end to at least aperture 146, and in some embodiments visual indicator extends from the distal end to a region proximal of aperture 146.

### Graduated Markings

According to another embodiment of the present invention, devices and methods are disclosed for measuring a tract length between two locations using a measurement indicator associated with one or more devices of the telescoping assembly during a TIPS procedure. Two devices of the telescoping assembly may be used to measure the distance between two locations, for example the distance between two blood vessels. The relative distance between the distal ends of two devices corresponds to the relative distance of the proximal ends of the two devices. Therefore, once aligned, moving the proximal end of one device allows the distance between the distal ends of the two devices to be measured.

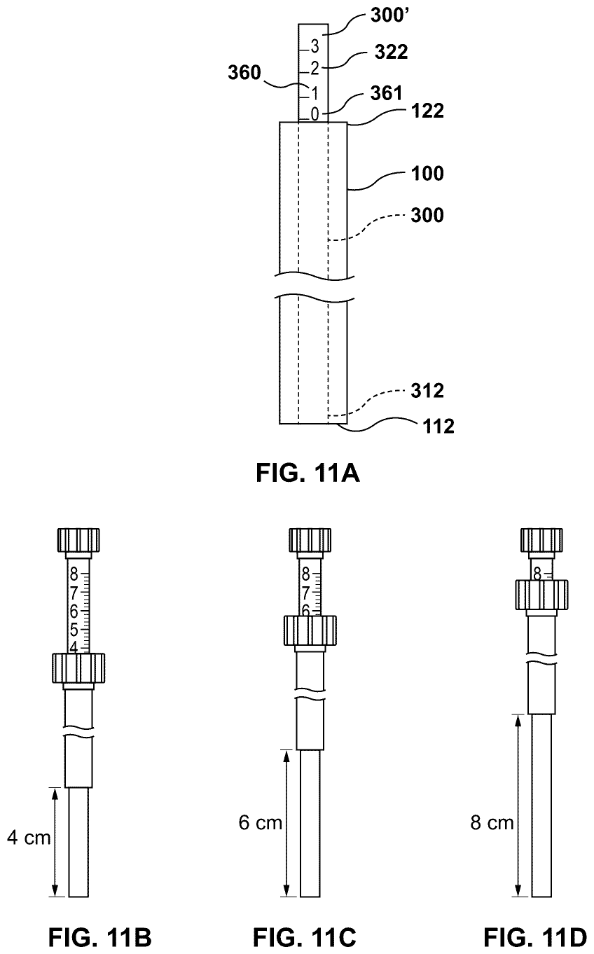

Referring to FIGS. 11A to 11D, in one embodiment, catheter 300′ is shown received within sheath 100. The proximal region of catheter 300′ (i.e., near the proximal end 322) comprises a measurement indicator 360. Measurement indicator 360 comprises at least one marking so that the proximal end of the outer device may be aligned with a “starting position” with respect to the inner device. The distance the proximal end travels with respect to the starting position can be used to determine the distance the distal end has traveled. In other words, the distal end of the outer device (sheath 100) is retracted until it reaches a “retracted position”, and the distance between the distal ends is equal to the distance between the starting position and the retracted position.

In one embodiment, measurement indicator 360 comprises graduated markings 361, i.e. a series of lines representing units of measurement (e.g. centimeters, millimeters, etc.). When the distal end 112 of the sheath 100 is aligned with the distal end 312 of catheter 300', as shown in FIG. 11A, the proximal end 122 of sheath 100 is located at a measurement marking corresponding to a starting position of the measurement indicator 360, for example a measurement marking of zero (0). In use, measurement indicator 360 is positioned on the proximal end 322 such that it is always located outside of a patient's body during a medical procedure.

Additionally, in order to visualize the distal end 112 under imaging, sheath 100 comprises a visual indicator 128 as described herein, which may be one or more marker bands 130 such as radiopaque bands or radiopaque ink. In other embodiments, sheath 100 body comprises a radiopaque material.

FIG. 11A shows the distal ends 112 and 312 of the sheath 100 and catheter 300′ respectively when the distal ends of each device are aligned, for example starting position is zero (0). As the sheath 100 is retracted proximally, shown in FIGS. 11B to 11D, sheath proximal end 122 moves and reaches a retracted position, for example 8 cm as shown in FIG. 11D, and the distance between the proximal ends corresponds to the distance between the distal ends. In other words, using measurement indicator 360, the distance between the distal ends is equal to the distance between the starting position and the retracted position.

With the catheter distal end 312 located in a first blood vessel, and the sheath distal end 112 located in a second blood vessel, the tract length (i.e., distance between the two blood vessels) can be measured using measurement indicator 360. In other words, with specific reference to a TIPS procedure, distal ends 112 and 312 start in the portal vein 12. Sheath 100 is retracted, through the tract, until the distal end 112 is located in the hepatic vein 10. The distance the sheath distal end 112 traveled is the tract length.

In other embodiments, distal end 112 and distal end 312 do not need to be aligned to determine a starting position on the proximal ends. Distal end 112 may be positioned proximal to distal end 312.

In other embodiments, catheter 300′ may be used combination with either of sheath 100 or sheath 100′. Further, contrast fluid may be injected through any device described herein in order to visualize the tract and/or blood vessels when determining a tract length using the measurement indicator 360, as described in method 1100 herein.

For clarification, with reference to a TIPS procedure, measuring a tract length means measuring a tract from the portal vein 12, to a region within the hepatic vein 10, which may be the HV/IVC confluence, or the tract may extend into the IVC.

### Method of Injecting Contrast and Measuring a Tract Length

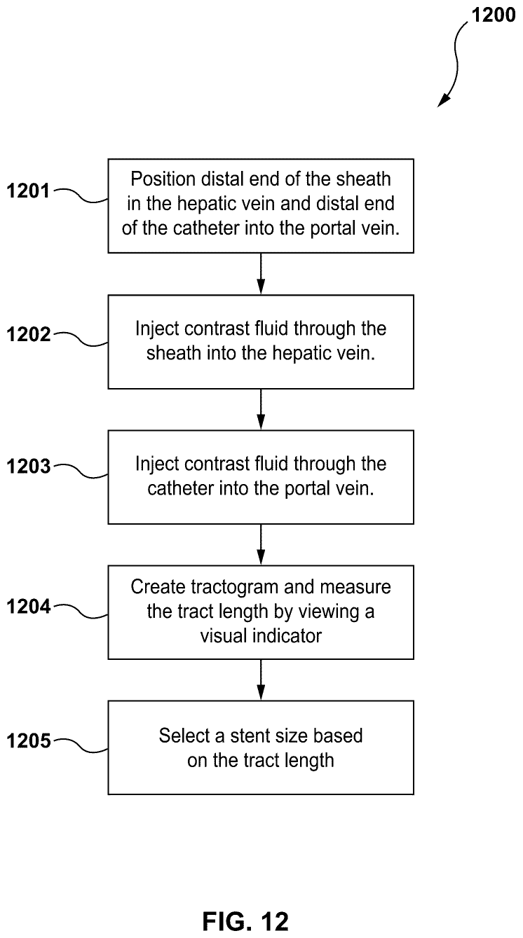

In order to select an appropriately sized stent for a TIPS procedure, the length of the tract from the hepatic vein to the portal vein must be determined. FIG. 12 shows a method of measuring a tract length that includes injecting contrast at two locations using multiple elongate devices arranged in a telescoping assembly 1000. This method forms part of a larger TIPS procedure.

Method 1200 begins at step 1201, after the puncture device 400 has punctured through the liver 30 and a tract has been created. The distal end of the sheath 100 is positioned in the hepatic vein 10 (or near the HV/IVC confluence), and the distal end of the catheter 300 is positioned in the portal vein 12. In this example, the dilator 200 has been withdrawn from the telescoping assembly 1000.

At step 1202, contrast fluid is injected through the primary lumen 102 of the sheath 100 into the hepatic vein 10, as previously shown in FIG. 7.

At step 1203, contrast fluid is injected through the primary lumen 302 of the catheter 300 into the portal vein 12. Steps 1202 and 1203 may happen concurrently. Contrast fluid may be injected through catheter 300 while the puncture device 400 is still within catheter primary lumen 302. Puncture device 400 may act as a guidewire during some or all portions of the TIPS procedure, or the puncture device 400 may have been removed from the assembly and replaced with a guidewire.

At step 1204, a tractogram is created to visualize the tract through the liver, from the hepatic vein 10 and the portal vein 12. A visual indicator is then viewed in order to measure the tract length between the hepatic vein 10 and the portal vein 12. For example, the visual indicator may be radiopaque marker bands 330 located on the catheter 300, and the tract length is determined by counting the number of marker bands.

At step 1205, an appropriate stent size is selected based on the measurement in the previous step. Other steps of the TIPS procedure may then be performed.

### Method of Injecting Contrast Using a Single Device to Measure a Tract Length

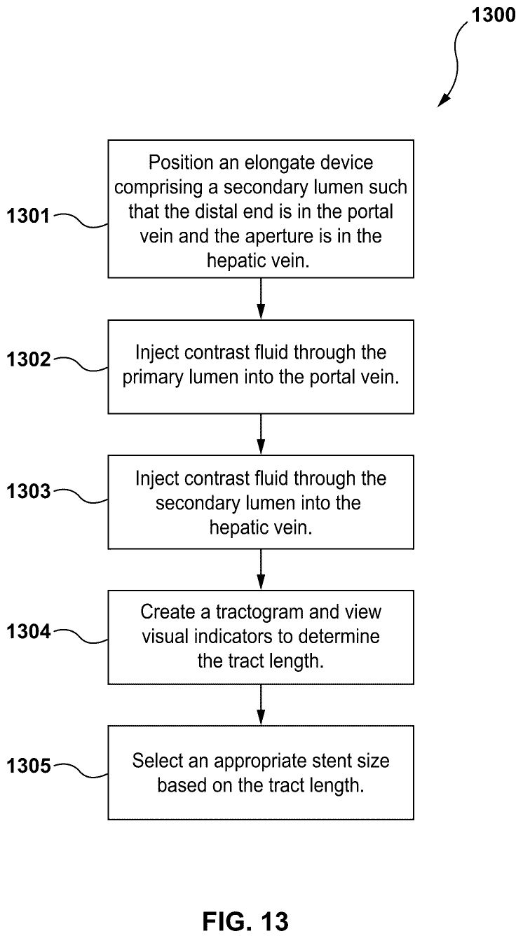

Referring to FIG. 13, a method of measuring a tract length that includes injecting contrast at two locations using a single elongate device is shown. This method may form one part of a TIPS procedure.

Method 1300 begins at step 1301, where sheath 100′, comprising an aperture, is positioned such that the distal end of the sheath 100′ is located in the portal vein 12 and the aperture is located in the hepatic vein 10.

At step 1302, contrast fluid is injected through sheath primary lumen 102, exiting at the distal opening 110 and into the portal vein 12.

At step 1303, contrast fluid is injected through secondary lumen 144, exiting at aperture 146 into the hepatic vein 10. This is similar to what is shown in FIG. 10. Steps 1302 and 1303 may happen concurrently.

At step 1304, a tractogram is created to visualize the tract. A visual indicator 128 located on sheath 100′ may be viewed. The visual indicator may optionally be located on a second device, for example, catheter 300, which is received within sheath 100′. The visual indicator may be used to measure the tract length between the hepatic vein 10 and portal vein 12, for example by counting the number of radiopaque marker bands.

At step 1305 an appropriate stent size is then selected based on the measurement determined in the previous step. Other steps of the TIPS procedure may then be performed.

### Method of Measuring a Tract by Retracting Telescoped Devices

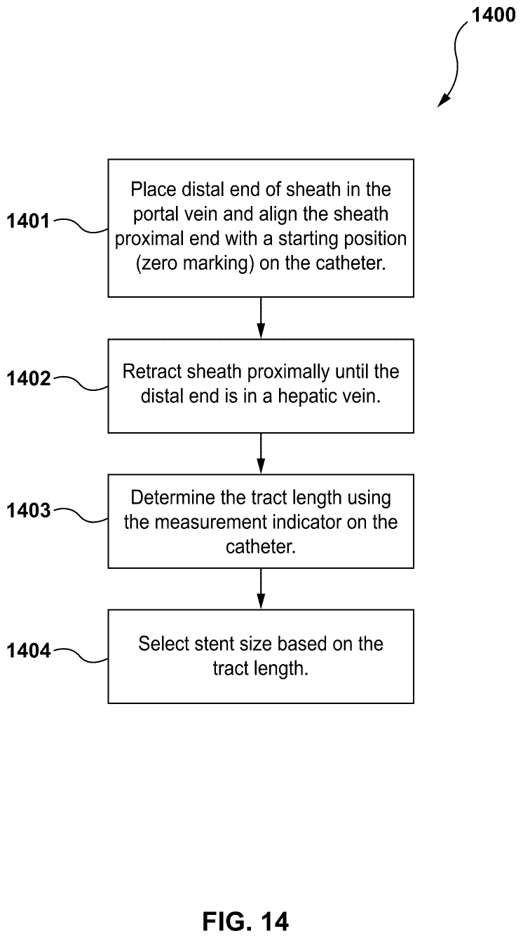

FIG. 14 shows an alternative method of measuring a tract length using telescoped devices comprising a measurement indicator. This method may form part of a larger TIPS procedure.

Method 1400 begins at step 1401, sheath 100 and catheter 300′ are arranged in a telescoping assembly and the distal ends 112 and 312 are aligned and positioned in portal vein 12. Contrast fluid may be injected into the tract and surrounding blood vessels at this step or any at another step.

Catheter 300′ comprises a measurement indicator 360 that defines a starting position on the proximal region. The sheath proximal end 122 is aligned with a portion of the measurement indicator 360, i.e., a starting position. For example, sheath proximal end 122 is aligned with a ‘zero’ marking.

At step 1402, the sheath 100 is retracted proximally until the distal end 112 is located in the hepatic vein 10. Once in the hepatic vein 10, the sheath 100 has reached a retracted position. Contrast fluid may be injected through the sheath 100 and/or catheter 300′ to visualize at least one of the blood vessels and/or tract.

At step 1403, the tract length between the hepatic vein 10 and the portal vein 12 is determined using the measurement indicator 360. The tract length is equal to the distance between the starting position and the retracted position.

At step 1404, a stent size is selected based on the tract length determined in the previous step. Other steps of a TIPS procedure may then be performed.

Method 1400 may optionally be used in combination with method 1200 or method 1300 to measure the tract length using the measurement indicator, or to confirm the tract length measured using the visual indicator.

The embodiments of the invention described above are intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

We claim:

1. A method of measuring a tract length during an intrahepatic portosystemic shunt procedure, the method comprising the steps of:
visualizing a tract between a first blood vessel and a second blood vessel by injecting contrast fluid into at least one of the first blood vessel and the second blood vessel using an elongate device, wherein contrast fluid injected into the first blood vessel is injected through a first opening of the elongate device and contrast fluid injected into the second blood vessel is injected through a second opening of the elongate device; and determining the tract length using a visual indicator.

2. The method of claim 1, wherein the step of visualizing the tract comprises injecting contrast fluid into the first blood vessel and the second blood vessel substantially concurrently.

3. The method of claim 1, wherein the visual indicator is associated with the elongate device.

4. The method of claim 1, wherein the visual indicator is associated with a second elongate device, the second elongate device being received within and moveable relative to the elongate device.

5. The method of claim 1, wherein the visual indicator comprises a plurality of radiopaque marker bands.

6. The method of claim 1, further comprising a step of selecting a stent suitable to be inserted between the first blood vessel and the second blood vessel based on the tract length.

7. The method of claim 1, wherein the elongate device comprises an elongate member defining a primary lumen and a secondary lumen, wherein the primary lumen terminates at the first opening which is defined by an inner wall and located at a distal end of the elongate member, and wherein the secondary lumen terminates at the second opening which is defined by an outer wall and located a linear distance proximal to the first opening.

8. The method of claim 4, wherein the visual indicator is associated with a proximal end of the second elongate device, and wherein the step of determining the tract length comprises moving the elongate device, while substantially maintaining a position of the second elongate device, and measuring a distance the proximal end of the elongate device has moved relative to the visual indicator.

9. The method of claim 1, wherein the first blood vessel is a portal vein and wherein the second blood vessel is a hepatic vein.

10. A method of measuring a tract length during an intrahepatic portosystemic shunt procedure using a first elongate device and a second elongate device, the second elongate device being received within and moveable relative to the first elongate device, the method comprising the steps of:
positioning a distal end of the first elongate device in a first location within a patient's body;
determining a starting position of a proximal end of the first elongate device relative to a proximal region of the second elongate device;
while substantially maintaining a position of the second elongate device, retracting the distal end of the first elongate device to a second location such that the proximal end moves to a retracted position; and
determining the tract length based on a distance between the starting position and the retracted position of the proximal end of the first elongate device.

11. The method of claim 10, wherein the proximal region of the second elongate device comprises graduated markings.

12. The method of claim 10, further comprising the step of injecting contrast fluid at least at one of the first location and the second location.

13. The method of claim 12, wherein the first location is a first blood vessel, and the second location is a second blood vessel.

14. The method of claim 13, wherein the first blood vessel is a portal vein, and wherein the second blood vessel is a hepatic vein.

15. A medical device operable to inject fluid, the medical device comprising an elongate member, the elongate member comprising:
an inner wall and an outer wall;
a first opening defined by a distal end of the elongate member;
a primary lumen defined by the inner wall and terminating at the first opening;
a second opening defined by the outer wall and located proximally of the first opening;
a secondary lumen terminating at the second opening; and
a visual indicator located at least between the first opening and the second opening;
wherein, in use, fluid injected through the primary lumen exits from the first opening and fluid injected through the secondary lumen exits from the second opening.

16. The medical device of claim 15, wherein the secondary lumen is defined between the inner wall and the outer wall.

17. The medical device of claim 15, wherein the secondary lumen is located substantially within the primary lumen.

18. The medical device of claim 15, wherein the visual indicator comprises a plurality of radiopaque marker bands.

19. The medical device of claim 15, wherein the visual indicator comprises radiopaque ink.

20. The medical device of claim 15, wherein the second opening is located between about 3 and about 7 centimeters from the first opening.
